(19) United States
(12) Patent Application Publication
(10) Pub. No.: US 2024/0050279 A1
(43) Pub. Date: Feb. 15, 2024

(54) SOUND ATTENUATION RATING SYSTEMS AND METHODS

(71) Applicant: 3M INNOVATIVE PROPERTIES COMPANY, St. Paul, MN (US)

(72) Inventors: Jeffrey L. Hamer, Springville, IN (US); Magnus S. Johansson, Jönköping (SE); Cameron J. Fackler, Indianapolis, IN (US)

(21) Appl. No.: 18/257,901

(22) PCT Filed: Dec. 8, 2021
(86) PCT No.: PCT/IB2021/061467

§ 371 Date: Jun. 16, 2023

Related U.S. Application Data
(60) Provisional application No. 63/199,283, filed on Dec. 17, 2020.

(51) Int. Cl. A61F 11/14; H04R 1/10; G10K 11/178

(52) U.S. Cl. CPC A61F 11/145; H04R 1/1008; H04R 1/1083; H04R 1/1041; G10K 11/17823; G10K 11/17873; H04R 2430/01; H04R 2460/15; H04R 2460/01; G10K 2210/3027; G10K 2210/1081; G10K 2210/3028

(57) ABSTRACT

A hearing protection assessment module for a Personal Protective Equipment (PPE) device. The module includes an ambient sound receiver that receives an indication of an ambient sound. The module also includes an experienced sound receiver that receives an indication of an experienced sound, wherein the experienced sound is a sound experienced by a user while wearing the PPE in the environment with the ambient sound. The module also includes a Field Attenuation Rating (FAR) calculator that, based on the ambient and attenuated sound indications, calculates a personal attenuation rating for the PPE device.

### BACKGROUND

The use of hearing protection devices (HPDs) and noise attenuating devices are well known, and various types of devices have been considered. Such devices include in-ear devices, such as earplugs, and over-the-ear devices, such as ear muffs, ear defenders, etc. Performance of hearing devices has often been evaluated in a laboratory environment. According to one method, an artificial test head or dummy head having artificial ear canals with heating simulated flesh leading to a microphone acting as an ear drum, may be used, in combination with another microphone outside the device.

### SUMMARY

A hearing protection assessment module for a Personal Protective Equipment (PPE) device. The module includes an ambient sound receiver that receives an indication of an ambient sound. The module also includes an experienced sound receiver that receives an indication of an experienced sound, wherein the experienced sound is a sound experienced by a user while wearing the PPE in the environment with the ambient sound. The module also includes a Field Attenuation Rating (FAR) calculator that, based on the ambient and attenuated sound indications, calculates a personal attenuation rating for the PPE device.

The above summary is not intended to describe each disclosed embodiment or every implementation. The Figures and Detailed Description, which follow, more particularly exemplify illustrative embodiments.

### BRIEF DESCRIPTION OF DRAWINGS

The present description will be further explained with reference to the appended Figures, wherein like structure is referred to by like numerals throughout the several views, and wherein:

FIGS. 1A and 1B are schematic diagrams of exemplary prior art systems for assessment of an over-the-ear hearing device.

FIG. 2 is a schematic illustration of hearing protection functionality and evaluation.

FIGS. 3A-C illustrates hearing protection devices providing Personal Attenuation Ratings for a user in accordance with embodiments herein.

FIG. 4 illustrates a schematic of a hearing protection system in accordance with embodiments herein.

FIG. 5 illustrates a method of providing in-situ field attenuation values for a wearer of a hearing protection device.

FIG. 6 illustrates an industrial environment in which systems and methods herein may be useful.

FIG. 7 illustrates a field attenuation monitoring system built into a hood.

FIGS. 8-10 illustrate example devices that can be used in the embodiments shown in previous Figures.

While the above-identified figures set forth various embodiments of the disclosed subject matter, other embodiments are also contemplated. In all cases, this description presents the disclosed subject matter by way of representation and not limitation. It should be understood that numerous other modifications and embodiments can be devised by those skilled in the art which fall within the scope and spirit of the principles of this description.

### DETAILED DESCRIPTION

The present description provides hearing protection devices and methods of evaluating performance of hearing devices in-situ by measuring, while a user is wearing the hearing protection device. This allows for a field attenuation rating (FAR) to be calculated for an individual, while they are wearing a given hearing protection device, in the field. Since many factors can affect the performance of a hearing protection device in-situ, including fit, proper environmental sealing, ear canal shape and size, as well as activity which may jostle a hearing protection device, it may be useful to have the ability to determine a current FAR for an individual.

Prior Art systems and methods allow for an acoustic assessment to be performed while a device is in a normal position of use, and, for example, allows an assessment to be performed at various times during the life of the hearing device, for example as described in U.S. Pat. No. 10,617,333, issued on Apr. 14, 2020, which allowed assessment of the device while it is in a position of use on a user. However, prior art devices have not provided a method or system that allows for real-time, in-situ measurement of a real-time attenuation for an individual wearing a hearing protection device in the field.

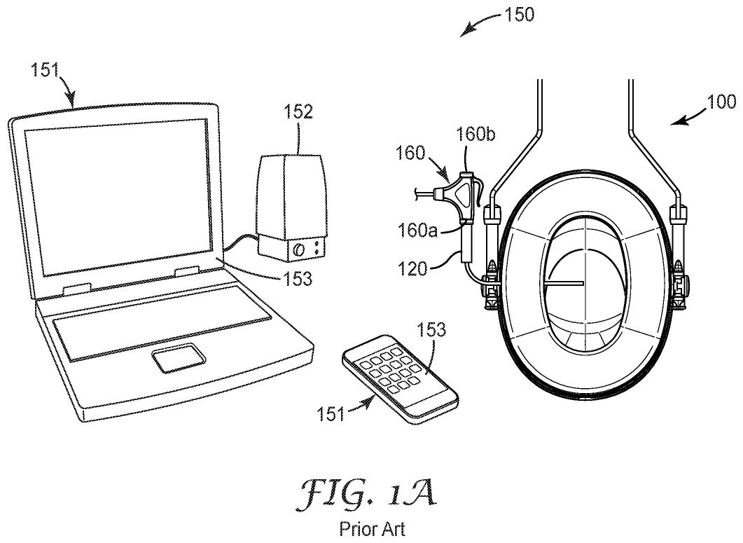

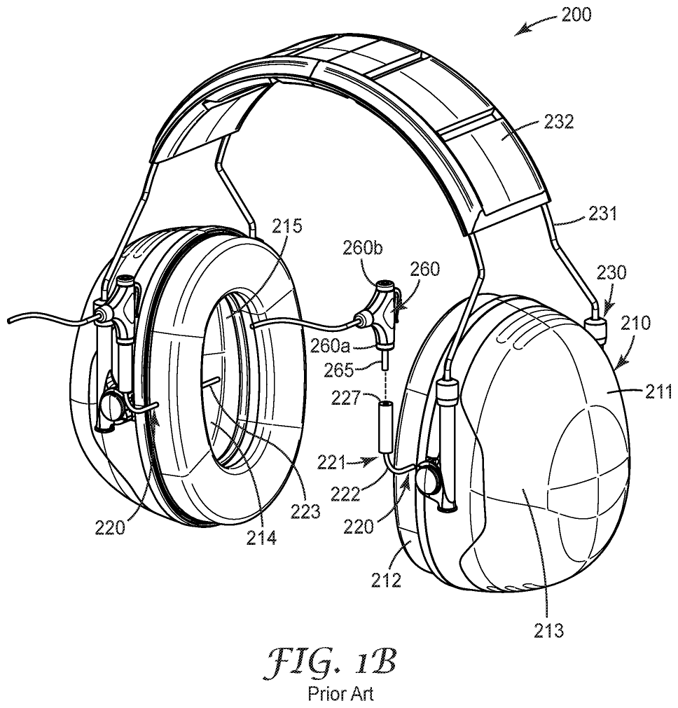

FIG. 1A shows a Prior Art over-the-ear hearing protection assessment apparatus 150 for assessing acoustical performance of an over-the-ear hearing device 100, as described herein. Over-the-ear hearing device 100 may be an earmuff, ear defender, communication device, hearing protection device, or other over-the-ear hearing device. Hearing device 100 includes an acoustic port 120 that extends through hearing device 100 and provides a passageway between an exterior and an interior volume of hearing device 100.

In an exemplary embodiment, apparatus 150 includes a controller 151, operatively connected to a broadband reference sound source 152, and a user interface 153. Sound source 152 is configured to generate a sound having a frequency range between 100 Hz to about 10000 Hz, and includes such octave bands of 125 Hz, 250 Hz, 500 Hz, 1000 Hz, 2000 Hz, 4000 Hz, and 8000 Hz, for example. Controller 151 is configured to provide a signal to control operation of sound source 152. In an exemplary embodiment, controller 151 is a computing device having a processor, such as a personal computer, smart phone, handheld device, dedicated controller, or other suitable controller as known in the art. One or more user interface components 153 are operatively connected to controller 151 such that a user may operate apparatus 150, and may include any suitable user interface components for a user to provide input and receive output, such as a keypad, keyboard, touch screen, voice input, speaker, display, connection, other suitable user interface components as known in the art, and combinations thereof. Controller 151, sound source 152, user interface components 153, and/or other components of apparatus 100 may be housed separately or housed together as a “stand-alone” over-the-ear hearing protection assessment apparatus, for example.

Apparatus 150 includes one or more microphones 160 operatively connected to controller 151. In an exemplary embodiment, a first microphone 160a is positioned in fluid communication with acoustic port 120 of over-the-ear hearing device 100 to detect an interior sound level and a second microphone 160b is positioned to detect an exterior sound level proximate to the over-the-ear hearing device 100. First and second microphones 160a, 160b, may be separately mounted microphones or may share a common housing, or otherwise be joined and/or mounted together, for example in back-to-back relationship relative to one another such that first microphone 160a measures a sound level at an interior volume of over-the-ear device and second microphone 160b measures an external sound level proximate over-the-ear device 100 while device 100 is in a position of use at least partially over an ear of a user.

Over-the-ear hearing devices providing a level of hearing protection as described herein may be assessed to obtain an in-situ indication of real-time attenuation provided, herein referred to as a Field Attenuation Rating (FAR). As described herein, a FAR may include a number of different calculations and corrections, including delivering a PAR value, as explained in ANSI draft standard BSR/ASA S12.71 “Performance criteria for systems that estimate the attenuation of passive hearing protectors for individual users,” for example. A PAR is a single number value that represents the individual attenuation that a user would obtain in a Real-Ear Attenuation at Threshold (REAT) test performed in a laboratory setting, for example, that may be performed as known in the art, and is representative of an best case or average attenuation obtained by a user of a particular hearing protection device, represented by the NRR label values in the United states and countries aligned with the US standard or the SNR for Europe and countries aligned with the EN standards. A user's PAR may be obtained from a measured noise reduction in which an interior sound level and exterior sound level are compared, where the interior sound level refers to a sound captured under a hearing protection device. Exemplary hearing protection devices as described further herein allow consistent and repeatable detection of interior and exterior sound pressure levels while the device is worn by a user and facilitate determination of a user's Personal Attenuation Rating (PAR) for a particular hearing protection device. A PAR value is one example of a variety of FAR values that may be calculated for a user, as described herein. For example, a simple field attenuation indication may be calculated by subtracting an interior sound level from an exterior sound level to capture an indication of attenuation currently provided by the hearing protection device.

FIG. 1B shows an exploded view of a Prior Art over-the-ear hearing protection device 200 including an ear cup 210 having a shell 211 and a cushion 212, and an attachment portion 230 having an arm 231 and a headband 232. Ear cup 210 is joined to headband 232 by arms 231, and may be supported on the head of a user by headband 232. Ear cup 210 is positioned at least partially over an ear of a user in a position of use. Cushion 212 forms an acoustic seal with the head of a user to block external sound waves from entering a user's ear canal. Ear cup 210 is generally cup-shaped having an exterior surface 213 and defining an interior volume 214. A user's outer ear, or Pinna, may generally reside in the interior volume 214 when over-the-ear hearing device 200 is positioned for use, and cushion 212 at least partially encircles the ear of a user.

Ear cup 210 is a sound attenuating ear cup and includes an acoustic attenuating material 215 that damps sound waves and/or attenuates sound waves entering the interior volume 214. Shell 211 may be formed from any suitable material including acrylonitrile butadiene styrene (ABS), polypropylene other suitable materials, and combinations thereof, for example.

Over-the-ear hearing device 200 includes a cushion 212 attached to shell 211. Cushion 212 forms an acoustic seal around an ear of a user and also distributes the pressure exerted by hearing device 100 against the user's head to promote comfort. Cushion 212 includes a mating feature that may be joined to a complementary mating feature of shell 211.

Headband 230 is generally “U-shaped” and sufficiently resilient to fit to the head of a user. Ear cup 210 may be attached to a helmet or other head covering, or supported by other suitable bands passing around a rear of a user's head, as known in the art.

Over-the-ear hearing device 200 receives a sound sensor such as a microphone or other suitable instrument for detecting a sound pressure. When in fluid communication with tube 221, microphone 260 may be used to detect a sound pressure associated with a sound pressure at interior volume 214 of ear cup 210 while over-the-ear hearing device is in a position of use, as described further herein.

Microphone 260 may include first and second microphones 260a, 260b, such that first microphone 260a is in fluid communication with acoustic port 220 of over-the-ear hearing device 200 to detect an interior sound pressure level and a second microphone 260b is in fluid communication with an exterior sound pressure level proximate over-the-ear hearing device 200.

An over-the-ear hearing device assessment apparatus having an acoustic port 220 extending through a removably attachable cushion 212 allows acoustic access to interior volume 214 without interfering with a fit of over-the-ear hearing device 200 on a user, for example because no wires or other components are required to pass between cushion 212 and a user's head. Furthermore, an assessment may be performed on the hearing device unit worn by a particular user, outside of a laboratory setting, if desired, and may be performed periodically over the life of the unit simply by attaching cushion 212. Accordingly, an attenuation value, such as a field attenuation rating (FAR), may be determined that is highly representative of an attenuation experienced by a user when wearing a particular over-the-ear hearing device 200 in the field.

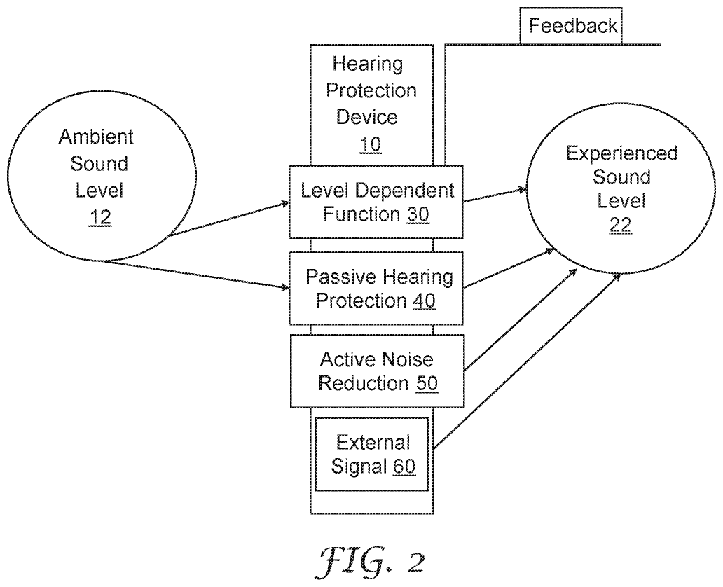

FIG. 2 illustrates the functionality and effectiveness of a hearing protection device 10. An ambient sound level 12 is present on an exterior of a hearing protection device 10. Hearing protection device 10 may be earplugs, earbuds, earmuffs, part of a hood or helmet or another suitable PPE device that includes at least one of the illustrated functionality. Hearing protection devices 10 includes at least some passive hearing protection 40, resulting from the device in combination with other sound absorbing materials such as foam or other acoustic insulation and a seal that separates a user's ear from ambient sound 12. In addition to passive hearing protection 40, hearing protection device 10 may also have some level dependent functionality 30 that can reduces a sound pressure level of an ambient sound 12 to a safe level and broadcasting this safe sound to the user under the hearing protector. For example, an ambient sound may have an unsafe sound pressure level of 90 dB, and a level dependent function 30 may process the ambient sound to a safe level before broadcasting it to a user. Hearing protection device 10 may also include active noise reduction functionality 50, which includes electronics actively contributing to the total attenuation of the product by creating a sound pressure with opposite phase. A hearing protection device 10 may also receive an external signal 60, such as an incoming radio or other signal that is broadcast to a user.

Determining whether or not a device is fit correctly requires measuring the ambient sound level 12, which is captured by a microphone on an exterior of the hearing protection device 10, and an experienced sound level 22. The experienced sound level 22 is derived from a captured signal under the hearing protection device 10, adjusted for open ear condition at the ear drum, which is accomplished by translating the measured sound to what would be experienced by an open ear. The translation is a diffuse field correction, such that the sound is corrected to what the experienced sound would be in the same position if a wearer's head was not present.

Hearing protection devices 10 are often sold with a rating, such as a Noise Reduction Rating (NRR), which reflects an ideal attenuation of the device, when fit and used correctly by a user. For example, as illustrated in FIG. 2, an ambient sound level 12 is 90 dB, and a user wearing hearing protection device 10 experiences an experienced sound level 22 of 69 dB. Hearing protection device 10 may have an expected attenuation of 30 dB, e.g. an expectation that the 90 dB sound would be reduced to 60 dB. The actual attenuation experienced by the user, in real-time when exposed to the ambient sound level 12, a reduction of 21 dB, is referred to herein as a field attenuation rating, and gives important insight into whether a user has sufficient hearing protection. The field attenuation rating can be converted to a Personal Attenuation Rating (PAR) by compensating to estimate how the user would experience the same sound in a laboratory setting. This may be done, for example, using diffuse field correction techniques.

A FAR or PAR may be useful for a variety of reasons, including determining whether or not hearing protection device 10 is fit properly. For example, poorly fit earplugs leak, and detecting that a FAR differs from an expected attenuation by greater than a threshold amount (e.g. more than 5 dB) may indicate that a user is not wearing hearing protection device 10 correctly, that hearing protection device 10 has a leak, or otherwise is not functioning correctly. A FAR may also be used to develop trends over time. For example, an expected attenuation may be based on a previous FAR. Deviation may then be useful for determining that hearing protection device 10 is not working correctly or poorly fit in that instance.

In some embodiments herein, a FAR is calculated while level dependent function 30, active noise reduction 50 and an external signal 60 are all off, so that only an evaluation of passive hearing protection 40 is calculated. In other embodiments, one or more of level dependent function 30, active noise reduction 50 or an external signal are operating when a FAR is calculated, to measure a exposure with these other factors accounted for. In some embodiments, the FAR value is converted to a PAR value, which may also be stored and used for calculating worker safety trends over time.

However, while the systems of FIGS. 1A and 1B allow for repeat testing of a hearing protection unit, in a position of use, it does not allow for in-situ testing and evaluation of a FAR for a user while the device is worn in a noisy setting, such as that of FIG. 2. For example, from one day to another, a significant difference, for example as much as a five-decibel difference, or even higher, in FAR may be experienced by a user. Additionally, whether a user is wearing safety glasses or eyewear can cause leakage, changing the FAR value. Movement during an activity, such as walking, running, lifting, etc. can cause a change in the device fit and therefore a change in the current FAR value for a user. The presence of facial hair or long hair can impact a FAR value. Additionally, device functionality, such as worn out cushions, component failure or wear-down can alter the achievable FAR value. For in-ear hearing protection devices, a good fit is key to an acceptable FAR value—a poor fit will result in more leakage and a lower FAR. Similarly, worn out tips for an in-ear hearing protection device also can impact achievable FAR values. Additionally, the FAR value may change for in-ear and over the ear hearing protection each time a user removes and re-inserts an earplug or dons and doffs an earmuff hearing protection device as a result of changes in device fit.

Because a FAR value can change due to a variety of factors from use-to-use, and during a given use, it is important to be able to evaluate a FAR value while a user is wearing the device in a noisy setting—e.g. while the device is being used in situ in an industrial environment.

Relevant changes could be detected and reported to the user, for example via voice prompt, so that the user can take action to change device settings or fit in the environment to maintain protection. As an example, if the user completed an initial fit test and began work in a location with environmental noise levels of 95 dBA, and it was determined that the user moved to an area with environmental noise levels that are 10 dB higher, the user can recheck his or her FAR and, if an unsatisfactory value is returned, the user can adjust their PPE or exit the area to reduce the risk of hearing loss.

Additionally, systems and methods herein provide the ability to determine a baseline FAR performance, such as during an annual hearing protector usage training Subsequent FAR monitoring could compare performance in real use to this baseline measurement to monitor changes. This may also help in detecting whether additional fit or use training is needed for a given worker.

Even without an annual baseline measurement, a history of real-time FAR measurements allows for establishing a database of individual performance. These historical measurements could be used to establish a baseline, for example taking the lowest observed FAR value. Ongoing measurements could monitor for changes relative to the historical trends. An example of this might be a worker on the tarmac of an airport, who starts to use her hearing protection device during the summer months. In the winter she wears a hat under the product and is alerted to the decrease in FAR caused by the leakage created by the hat.

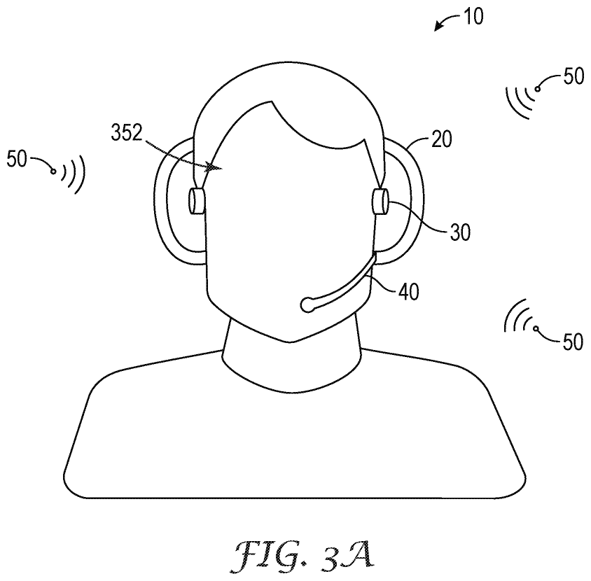

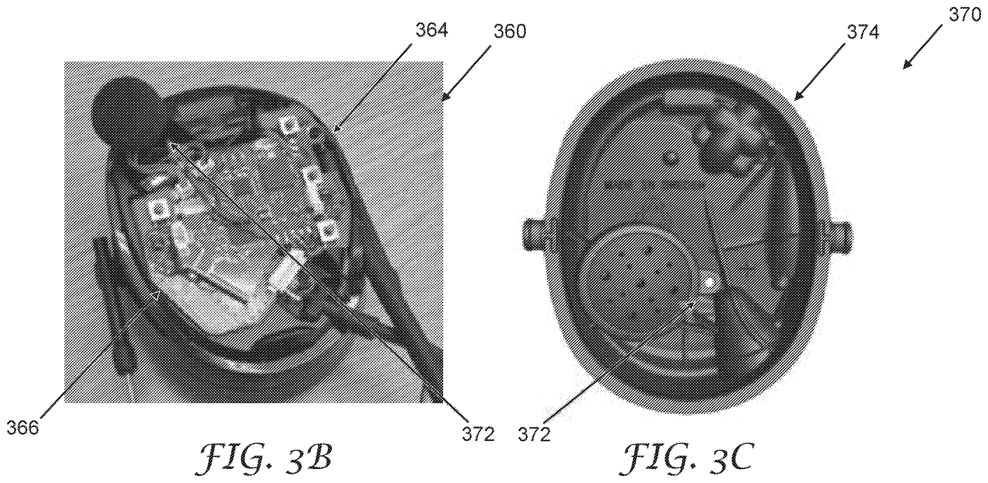

FIG. 3A illustrates a hearing protection system in accordance with an embodiment of the present invention. A person 310 may be in an environment with a plurality of sounds 350. Different sounds 350 may have different noise levels associated with them. Some of noises 350 may be safe to hear at ambient levels, while others are not.

In one embodiment, person 310 wears one or both of an over-ear hearing protection system 320 or an in-ear hearing protection system 330. Over-ear hearing protection system 320 is illustrated as a pair of earmuffs while in-ear hearing protection system 330 is illustrated as a pair of in-ear devices. However, it is expressly contemplated that both hearing protection systems 320, 330 are active hearing protection systems that include microphones to capture ambient sounds, attenuate the captured ambient sound to a safe level, and provide the sound to a user using a speaker positioned on an interior of the earmuff or within the ear canal, this is referred to as a Level Dependent Function (LDF).

While both first and second hearing protection systems 320, 330 are configured to operate as independent LDF hearing protection systems, they may also configured to enter a dual protection mode and coordinate hearing protection functionality. As envisioned herein, a FAR value may be obtained by either, or both, of hearing protection systems 320, 330. Dual hearing operation is described in greater detail in PCT Application with Ser. No. IB2020/059245, filed on Oct. 2, 2020. The obtained FAR value may be further transformed into a PAR value, as desired by a hearing protection wearer or safety officer for an industrial environment.

Hearing protection devices 320, 330 are each configured, when operating alone, to receive ambient sound 350 and, based on ambient sound 350, provide an amplified, similar or attenuated sound 352 to a user, depending on the device settings and exterior environmental sound pressure levels. The sound passed through the device 352 is provided could be based on an anticipated fit of hearing protection devices 320, 330. For example, if in-ear hearing protection device 330 is not properly fit within an ear canal of user 310, the sound passed through the device 352 may, in combination with leakage of sounds 50 into the ear canal, be high enough to cause hearing damage to the user 310.

Therefore, it is important for a hearing protection device 320, 330 to be configured to measure a FAR value in the environment of FIG. 3A. The FAR value may be used, for example by a safety officer, to determine whether a user has a sufficient seal or adequate fit. The ability to measure it in the environment of use is important, particularly with in-ear hearing protection devices which can have a higher fit variation for different individuals.

FIGS. 3B and 3C illustrate views of an external portion of an earmuff cup and an internal portion of an earmuff cup. As illustrated in external portion 360, a housing 364 partially encloses an exterior microphone 372, which may capture ambient noise. External microphone 360 may also capture a sound pressure level of ambient noise, in some embodiments. In other embodiments, the captured ambient noise is provided to a controller, for example a processor on a printed circuit board assembly (PCBA) 366.

On an internal portion 370, of an earmuff cup, an interior housing 374 separates an internal microphone 372 from an external environment, outside of the cup, such that microphone 372 substantially only picks up a sound pressure level, and/or captures a sound, within the earmuff housing.

In some embodiments, a seal is present between inner portion 370 and external portion 360, to reduce the possibility of ambient sound leakage from the ambient environment to the inner portion 370 and, subsequently, to a wearer of hearing protection device 300.

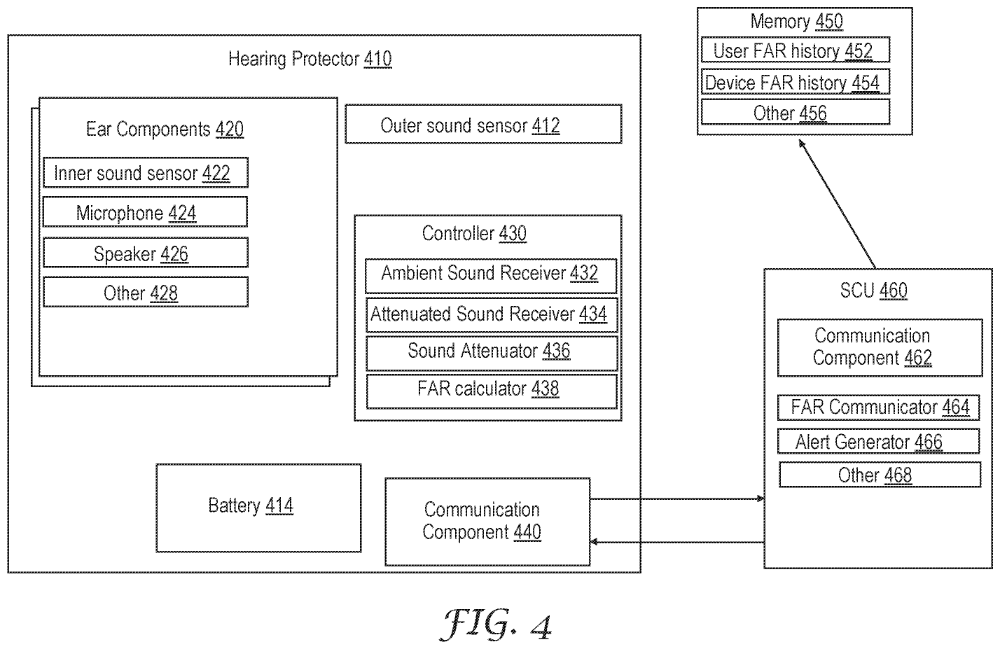

FIG. 4 illustrates a schematic of a hearing protection system in accordance with embodiments herein. Hearing protector 410 may communicate with a control unit 460, as illustrated in FIG. 4. However, in some embodiments, the components of control unit 460 are part of hearing protector 410. A memory 450 is illustrated in FIG. 4 as separate from hearing protector 410, for example as part of a central hub or a cloud storage. However, it is expressly contemplated that, in some embodiments, memory 450 is part of hearing protector 410, or is in direct communication with hearing protector 410.

Hearing protector 410 includes two hearing components 420 that may be either an in-ear hearing protection unit or an over-ear hearing protection unit. Hearing component 420 has one or more microphones 424 that capture ambient sound from an environment around hearing protector 410. A speaker 246 provides an amplified, similar or attenuated sound, depending on the device settings and the measured external microphone sound pressure level, to a user, and may, for example, be positioned within an earmuff cup or within a user's ear canal. Ear component 420 also includes an inner sound receiver 422, such as a microphone, configured to detect a sound level within the ear component. Inner sound receiver 422 senses a sound actually experienced by a user wearing hearing protector 410. Inner sound receiver 422 may measure an experienced sound in decibels, or in another suitable unit. Ear component 420 may include other features.

Hearing protector 410 also includes an outer sound receiver 412, which measures a noise level associated with ambient sound. Outer sound receiver 412 may be positioned near microphone 424, in some embodiments, or may be positioned in another suitable location associated with hearing protector 410 such that an accurate measurement of ambient sound close to the user's ears can be detected.

Ear component(s) 420 may also include other features 428. For example, ear components 420 may couple to each other with a headband.

Hearing protection unit 410 may also include a controller 430. Controller 430 is illustrated as part of hearing protector 410. However, in some embodiments, controller 430 is part of a control unit 460, or is communicably coupled to hearing protector 410.

Controller 430 includes an ambient sound receiver 432 that receives an indication of a sound from outer sound receiver 412. The indication may be an actual sound received, or just an indication of a sound, for example a detected decibel level of the ambient sound. Another suitable ambient sound indication may also be received from outer sound receiver 412.

Controller 430 also includes an attenuated sound receiver 434 that receives an indication from inner sound receiver 422. If hearing protector 410 is correctly fit and functioning as intended, the indication of the experienced sound level from inner sound receiver 422 should match an expected attenuated sound level. Any difference between the actual attenuated sound received by attenuated sound receiver 434 and that broadcast through speaker 426 may be an indication that ear component(s) 420 are not fit properly or not functioning properly. The actual experienced sound level is used to calculate a FAR, by FAR calculator 438, which may provide a numerical FAR rating, a deviation from the expected attenuation, a calculated PAR rating, or another suitable output, such as an alert of a potential malfunction. The FAR calculation may be exclusive, or inclusive, of level dependent or active noise reduction functionality that may be present in the device 410.

Speaker 426 broadcasts an attenuated sound to a wearer of hearing protector 410. Speaker 426 may be an acoustic, bone conduction or flesh conduction device. The attenuated sound may a sound modified by sound attenuator 436. The attenuated sound may be reduced, amplified, or cancelled. The sound may also be an added sound, such as an incoming radio broadcast, music playing, or voice, such as from an incoming call broadcast through ear component 426.

FAR calculator 438 calculates a personal attenuation rating based on the actual attenuation experienced by a user. The personal attenuation rating may be reported as an actual experienced FAR by the user, such as the number “21 dB”, referring back to FIG. 2, or a deviation from an expected FAR, e.g “+9 dB” from expected, or another suitable indicator, such as “Outside Acceptable Limits,” for example, if a 5 dB threshold for variation was set by a safety officer. The FAR may be communicated to a user of hearing protector 410, for example using a feedback mechanism of hearing protector 410, such as audio, visual or haptic feedback, or a feedback mechanism of control unit 460, such as audio, visual or haptic feedback. Additionally, in combination with knowledge of the external sound environment, the FAR calculation may be useful for augmenting a level dependent function, or active noise reduction functionality, in order to adjust for a detected leakage. For example, if the environment is 90 dB and the FAR is 25, a level dependent function may amplify reproduced sounds up by 10 dB such that a user experiences 75 dB. If the noise environment is 100 dB and the FAR is 15, active noise reduction functionality may be activated, or increased, so that the experienced sound level does not exceed 75 dB.

Hearing protector 410 includes a communication component 440, that allows hearing protector 410 to communicate with other devices. Communication component 440 may operate using wired or wireless communication protocols. As illustrated in FIG. 4, hearing protector 410 does not directly communicate with other personal protective equipment (PPE), with a hub or with cloud-based storage, but instead communicates directly only with a control unit 460. However, it is expressly contemplated that, in some embodiments, hearing protector 410 is also configured to directly communicate with other devices, using communication component 440. Communication component 440 may operate using any suitable wired or wireless protocols, including 802.11a/b/g/n AC or other IEEE 802.11 wireless protocol; Bluetooth®, including any 2.4 GHz wireless protocol such as lite, low energy, etc.; near-field communication protocols, RFID-based communication protocols, or other suitable protocols. Bluetooth®/ISM protocols may operate using 2400 to 2483.5 MHz. Wireless protocols may also include DECT on 1.9 GHz, 5.8 GHz or 900 MHz, WiFi on 2.4 or 5.8 GHz, or PMR frequency bands from 66-960 MHz, DMR or the RF range 30 MHz to 1 GHz.

Hearing protector 410 is powered by an electrical power source 414, such as disposable and rechargeable batteries or other means of generating sufficient power, in some embodiments. Power source 414 provides power to controller 430, microphone(s) 424, speaker(s) 426, and sensors 412, 422, 424.

It is important that sensors 412, 422 be low-power sensors, as power source power source 414 must be able to provide sufficient use life for hearing protector 410. Additionally, controller 430 must also run on provided power.

In some embodiments, hearing protector 410 is one of several PPE devices or communication devices worn by a user. A control unit 460 may be communicably coupled to hearing protector 410, as well as other devices. This may be useful for adaptive signal routing, for example. The control unit 460 may also provide additional functions that would not be possible given size constraints on a processor stored wholly within a hearing protector 410. For example, a level dependent function, two-way radio function, and signal routing to external devices. Additionally, the presence of a control unit 460 may allow for better user interface features such as pushable button, better access to battery charging, etc. In some embodiments, some or all components illustrated as performed by controller 430 are performed by a processor within control unit 460. For example, based on physical size limitations of in-ear hearing protection devices, it may be convenient to relay sensor signals from sensors 412, 422 to a processor of control unit 460, which includes FAR calculator 438.

Control unit 460 includes a communication component 462 that sends and receives information to and from hearing protector 410. For example, while hearing protector 410 may include a microphone capable of picking up a user's voice (not shown), a microphone of higher quality may be present on another PPE device the user is wearing. Control unit 460 may detect both hearing protector 410 and the other device on a personal area network and send a signal to hearing protector 410 to turn off its microphone.

Control unit 460 may have a FAR communicator 464 that communicates a signal to initiate a FAR measurement to controller 430, and may report a measured FAR value to a memory 450. Additionally, if the FAR value, or a further calculated PAR value exceeds or fails to meet a threshold, set by a manufacturer, the user, or a safety officer, an alert generator 466 may generate an audio, visual, or haptic feedback based alert. The alert may go to a user wearing hearing protector 410, a safety officer responsible for an industrial site, another nearby worker, or may be communicated to memory 450. Control unit 460 may have other functionality 468, such as a personal area network generator that is configured to recognize and incorporate hearing protector 410 into a personal area network for a user.

Memory 450 is illustrated in FIG. 4 as remote from hearing protector 410 and control unit 460. However, it is expressly contemplated that, in some embodiments, at least some components illustrated as part of memory 450 are stored instead within a memory component of hearing protector 410 or control unit 460.

Memory 450 may include a user FAR history 452, which includes historical FAR values calculated for the user. The historical FAR values 452 may be correlated with devices that the user wore, which may indicate whether an issue is present with a given device (e.g. a current FAR value is outside the norm for the user) or a consistent trouble with obtaining a proper fit (several devices reporting FAR values that stray from an expected attenuation level). Such history can be used to determine whether a user needs to undergo fit testing or training on proper use of PPE. Memory 450 may also include device FAR history 454, which includes historical FAR values for a given device. The historical device FAR values 454 may be used to detect when a device is not operating correctly. For example, an increase in deviation from an expected attenuation level may indicate that a cushion is wearing out or that another sound leakage cause may be present. Memory 456 may also include other information 456, such as historic alerts regarding a user, historic activity of a user in an industrial setting, a user identification number, device settings, use or device location and/or position within an environment, etc.

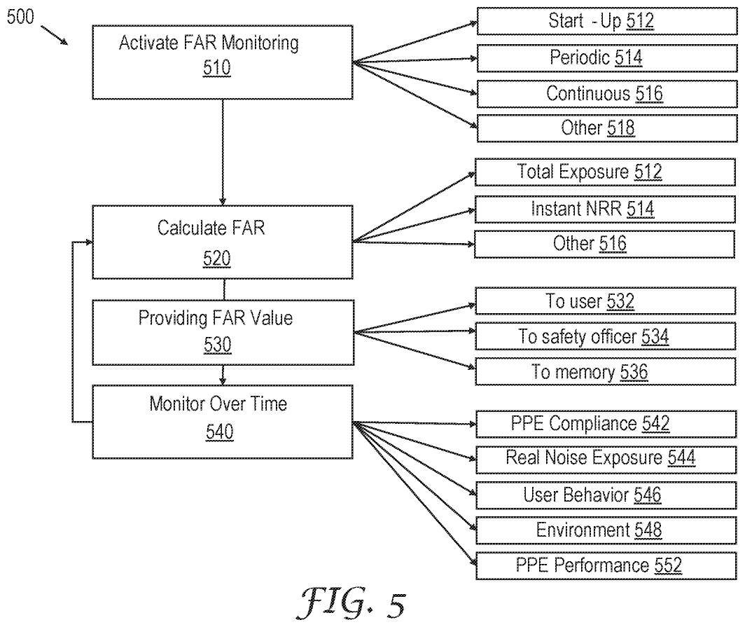

FIG. 5 illustrates a method of providing in-situ FAR values for a wearer of a hearing protection device. Calculating and monitoring how a FAR value for a user changes over time while a user is in an industrial setting can provide a lot of information that may be useful for monitoring individual health and device performance. For example, knowing that a FAR value for an individual correlates to a much lower FAR than expected may indicate to a safety officer that the individual needs to be retrained on safe use and proper fit of a hearing protection device. Additionally, monitoring use over time may indicate whether the device is functioning optimally. Method 500 provides a way to calculate and monitor a FAR value over time.

Method 500 may be implemented by a FAR calculation module within or associated with a hearing protection unit.

In block 510, FAR monitoring is activated. In order to conserve battery life, in some embodiments the components of an active fit testing system are either powered off or in a low power mode until activated. FAR monitoring should be initiated, as indicated in block 512, at start-up of a hearing protection device, or when a user is detected as wearing the device. FAR monitoring may also be initiated periodically, as indicated in block 514. In some embodiments, initiation of FAR monitoring may be required for communications functionality to work as communications may not be clear without first ensuring that a device is suitably fit to a user. Periodically may include activating a FAR monitoring module every hour, every 30 minutes, or at another pre-set interval during a shift. Periodically may encompass apparently ‘random’ checks to a user, e.g. at pre-set times selected by a controller or FAR monitoring unit, but not at regular intervals. FAR monitoring may also be done substantially continuously, as indicated in block 516. For example, sensors of a FAR module may report measured sound levels every second, or multiple times per second, to provide substantially real-time constant tracking of a user's FAR. Other triggers may also cause a FAR monitoring module to activate, as indicated in block 518. For example, an accelerometer may indicate that a hearing protection unit was dropped, or may have been jostled.

In block 520, a FAR value is calculated. A FAR calculation module includes an ambient sound receiver that measures the noise level of ambient sound, for example in decibels. The ambient sound receiver is located on an exterior of a hearing protection. The FAR calculation module also includes an attenuated sound receiver, located on an interior of the hearing protection device that measures the sound level as experienced by a user. For an over-the-ear headset, the attenuated sound receiver may be located on an interior of an ear-muff cup, or in another suitable position. For an in-ear plug, the attenuated sound receiver is positioned within the ear canal of a user, or in another suitable position. The FAR calculation module subtracts the signal of the attenuated sound receiver from the ambient sound receiver to determine the current FAR value for the device, as indicated in block 514. This can then be compared to a labeled NRR/SNR or other regulatory metric determined in a laboratory setting and provided with the product per regulatory requirements. The FAR calculation module may also be able to calculate a total noise exposure, as indicated in block 512. A total noise exposure may be extrapolated based on current FAR and a time that the hearing protection device has been worn in embodiments where the environmental sound levels are monitored/collected during the exposure. A PAR may also be calculated from the FAR value, for example by conducting field diffuse transformation. Additionally, other variations on the FAR calculation are envisioned, as indicated in block 516. For example, it is envisioned that, in many embodiments, that LDF or ANR functionality are not active when an FAR is calculated, such that only the passive attenuation ability of a device is evaluated. However, it is expressly contemplated that evaluation of LDF or ANR features may be assisted by including their functionality in an FAR calculation.

In block 530, the calculated FAR value is provided. For example, the FAR value, or a comparison to a threshold value, may be provided to a wearer of the hearing protection device, as indicated in block 532, through any suitable audio, visual or haptic feedback mechanism. The FAR value may also be provided to a safety officer of the site, as indicated in block 534. The FAR value may also be provided to memory, as indicated in block 536, in a format that can be retrieved for later analysis.

In block 540, the FAR module can continue to, while activated, to monitor the FAR value over time. For example, a series of FAR values may be calculated in a short period of time that the FAR module is active, such that an average can be taken. In embodiments where a FAR value is captured multiple times during a shift, the captured internal and external senor data can be analyzed to capture information about a worker and the work environment over time. For example, compliance with PPE rules may be monitored, as indicated in block 542. A real cumulative noise exposure may also be measured and tracked, as indicated in block 544. User behavior, such as proper wearing of PPE, different environments and noise reduction required during a work shift, may also be monitored, as indicated in block 546. Information about the environment may also be captured, such as where noise levels are higher and lower, as indicated in block 548. Additionally, performance and functionality of the hearing protection device can also be monitored, as indicated in block 552.

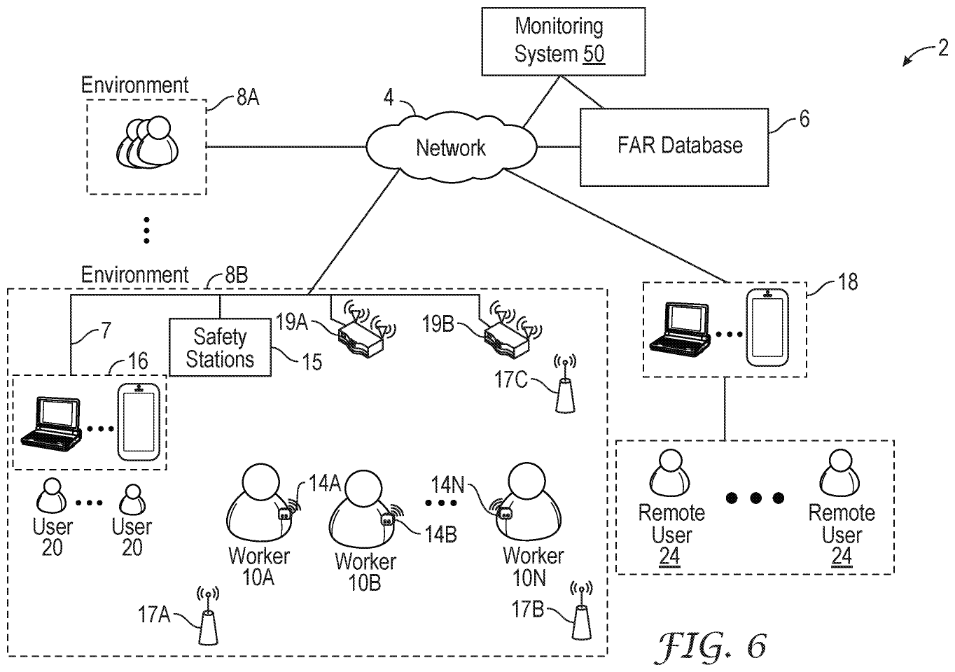

FIG. 6 illustrates an industrial environment in which systems and methods herein may be useful. FIG. 6 is a block diagram illustrating an example network environment 2 for a worksite 8A or 8B. The worksite environments 8A and 8B may have one or more workers 10A-10N, each of which may need to interact with equipment or environments that require the use of personal protective equipment such as glasses, hard hats, fall protection equipment, respirators, gloves, etc. Workers 10A-10N may have a range of experience with PPE usage. Some may have recently undergone fit testing, some may need training on proper use of hearing protection devices, and others may need retraining Additionally, different areas in environment 8B may be subject to different sound levels. For example, a cafeteria may generally not require hearing protection, while a loading dock or construction zone may require hearing protection.

Environment 8B may include a worker monitoring system 50 for detecting and managing worker safety. Monitoring system 50 may access an FAR database of captured FAR values for workers 10A-10N. FAR values may be captured for each of workers 10A-10N during a work shift, for example at start-up, periodically, continuously, or when triggered. A safety officer may use monitoring system to determine whether or not workers are safe. Captured FAR values may provide an indication that the safety officer can use to determine whether a given worker has adequate hearing protection at a given time. Monitoring system 50 may also help detect incidents of noncompliance by workers of PPE rules, reducing the risk of injury and increasing safety within a worksite 2. System 50 may also allow safety professionals to manage area inspections, worker inspections, worker health and safety compliance training.

In general, monitoring system 50, as described in greater detail herein, is configured to allow for review of FAR values captured by individual PPE devices worn by each of workers 10A-10N during work shifts. Additional data, such as noise levels throughout environments 8A and 8B, and fluctuation in worker and environment behavior throughout the day may also be captured. System 50 may be connected, through network 4, to one or more devices or displays 16 within an environment, or devices or displays 18, remote from an environment. System 50 may be able to provide alerts, or facilitate alerts by a safety officer, to any of workers 10A-10N if an unacceptable FAR value is detected, as well as provide feedback on types of PPE, PPE usage and proper fit that may be appropriate for a given situation.

In some examples, each of environments 8 include computing facilities, such as displays 16, or through associated PPEs, by which workers 10 can passively or actively interact with system 50. For example, a worker 10A may provide a FAR value captured during a work shift over network 4. For examples, environments 8 may be configured with wireless technology, such as 802.11 wireless networks, 802.15 ZigBee networks, and the like. In the example of FIG. 6, environment 8B includes a local network 7 that provides a packet-based transport medium for communicating with computing system 16 via network 4. In addition, environment 8B includes a plurality of wireless access points 19A, 19B that may be geographically distributed throughout the environment to provide support for wireless communications throughout the work environment.

As shown in the example of FIG. 6, an environment, such as environment 8B, may also include one or more wireless-enabled beacons, such as beacons 17A-17C, that provide accurate location information within the work environment. For example, beacons 17A-17C may be GPS-enabled such that a controller within the respective beacon may be able to precisely determine the position of the respective beacon. Alternatively, beacons 17A-17C may include a pre-programmed identifier that is associated in system 50 with a particular location. Based on wireless communications with one or more of beacons 17, or data hub 14 worn by a worker 10 is configured to determine the location of the worker within work environment 8B. In this way, FAR values reported to monitoring system may be stamped with positional information. This may be helpful in the event a supervisor or safety officer needs to respond to an indication of inadequate hearing protection.

In example implementations, an environment, such as environment 8B, may also include one or more safety stations 15 distributed throughout the environment. Safety stations 15 may allow one of workers 10 to check out articles of PPE and/or other safety equipment, verify that safety equipment is appropriate for a particular one of environments 8, and/or exchange data. For example, safety stations 15 may transmit alert rules, software updates, or firmware updates to articles of PPE or other equipment. Removal of a hearing protection device from a safety station 15 may trigger, for example, collection of a FAR value.

Techniques and components of this disclosure may improve the safety of workers within an environment by improving PPE compliance within the environment. Systems and methods herein may also provide general information about whether additional PPE training is needed for a worker or group of workers, based on detected patterns of noncompliance. Additionally, systems and methods herein can help workers within an environment look out for each other by seeing alerts concerning noncompliance.

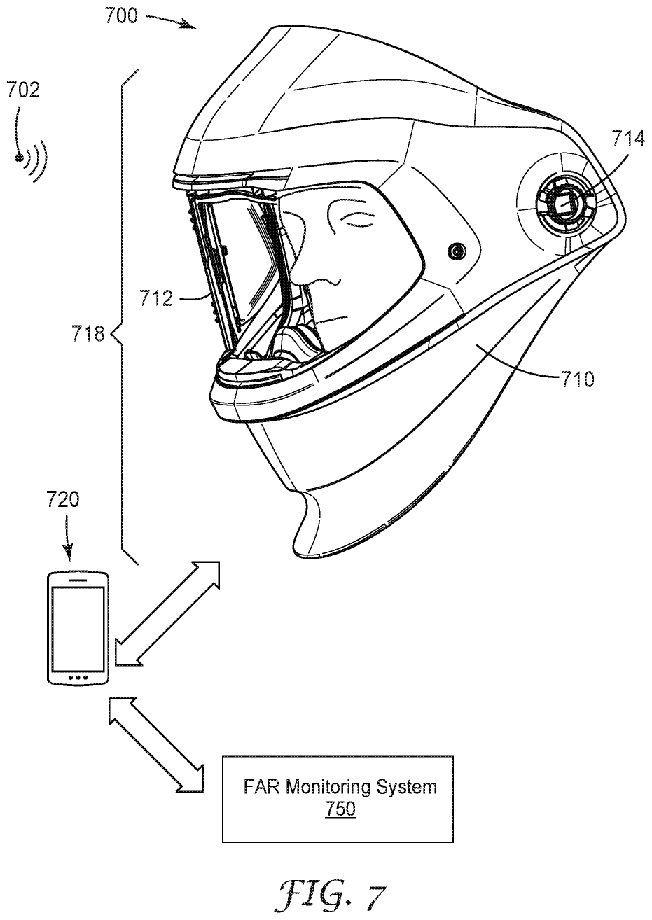

FIG. 7 illustrates a PPE device in which embodiments of the present invention may be useful. A FAR measurement system may be incorporated into any suitable PPE device that provides active hearing protection. For example, welding helmet 218, illustrated in FIG. 7, may be part of an active hearing protection system 700. Any PPE system that includes a microphone that picks up ambient noise 702, a processor to process the sound to a safe level, and a speaker that provides sound to a user may also include a FAR measurement system. For example, welding helmet 718 may include a built-in speaker, or may provide sound from a microphone to an in-ear speaker hearing protection unit, or an over-the-head hearing protection unit worn by a user under helmet 718.

Welding helmet 718 includes head-mounted device 710, visor attachment assembly 714 and one or more speakers (not shown) inside device 710 as well as one or more microphones (not shown) positioned on an exterior or interior surface of device 710, or on the outside of the attenuating part of the hearing protection device to capture external sounds.

As illustrated, PPE system 700 is in communicative contact with a separate device 720, illustrated in FIG. 7 as a cellphone, which may have an application through which a user or wearer of PPE system 700 may interact with a FAR monitoring application. However, it is expressly contemplated that, in some embodiments, a user may communicate directly with FAR monitoring database 750. Additionally, in some embodiments, a user of PPE system 700 may not directly interact with FAR monitoring system, and captured FAR information may not be available to a user, but only available to a safety officer or through a different dashboard (not shown) that interacts with FAR monitoring system 750.

In embodiments where a user of system 700 can receive data from FAR monitoring system 750, welding helmet 700 may include a screen 712 which may have augmented reality overlay abilities. A wearer may be able to, using audio, motion, or remote controller, interact with database 750 using screen 712. However, many PPE devices lack a screen and are designed to reduce processing power to preserve battery life. Therefore, in many embodiments, and as described herein, PPE devices are envisioned as interacting with database 750 using an intermediate device 720.

Additionally, while a cell phone 720 is illustrated in FIG. 7, it is expressly contemplated that other computing devices 720 are possible, including laptops, tablets, desktop computers, or other computing terminals able to interact, either in a wired or wireless capacity, with both of PPE device 700 and database 750. Additionally, a control unit, as described above with respect to FIG. 4, for example, may also be a suitable device 720.

Computing device 720 may generate any type of indication of output. In some examples, the indication of output may be a message that includes various notification data. Notification data may include but is not limited to: an alert, warning, or information message; a type of personal protective equipment; a worker identifier; a timestamp of when the message was generated; a position of the personal protective equipment; one or more light intensities, or any other descriptive information. In some examples, the message may be sent to one or more computing devices as described in this disclosure and output for display at one or more user interfaces of output devices communicatively coupled to the respective computing devices. In some examples computing device 720 may receive an indication of where a sound source originated (e.g. based on a communication from a device generating a recognized sound) and generate the indicated output further based on the sound source and sound type was occurring.

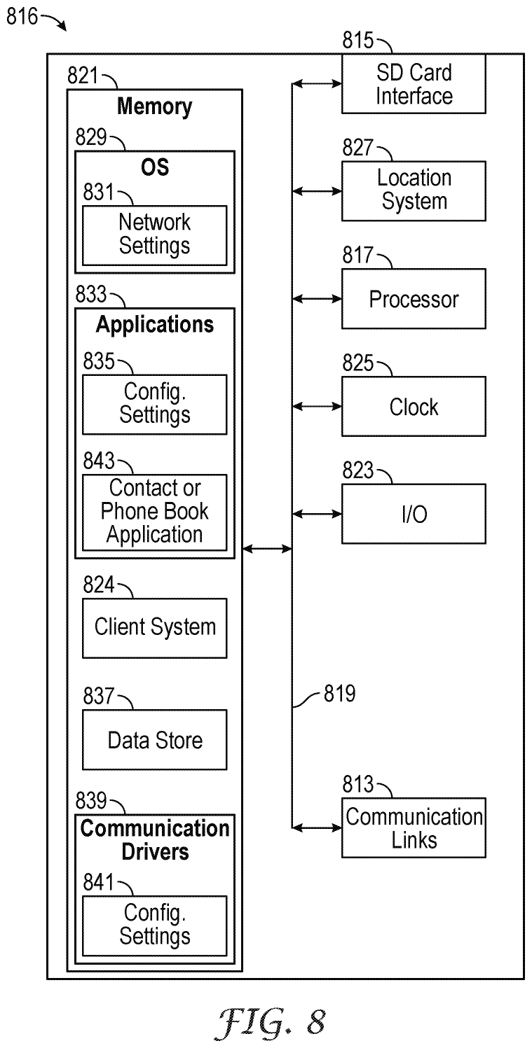

FIGS. 8-10 illustrate example devices that can be used in the embodiments shown in previous Figures.

FIG. 8 illustrates an example mobile device that can be used in the embodiments shown in previous Figures. FIG. 8 is a simplified block diagram of one illustrative example of a handheld or mobile computing device that can be used as either a worker's device or a supervisor/safety officer device, for example, in which the present system (or parts of it) can be deployed. For instance, a mobile device can be deployed in the operator compartment of computing device for use in generating, processing, or displaying the data.

FIG. 8 provides a general block diagram of the components of a mobile computing device 816 that can run some components shown and described herein. For example, mobile computing device 816 may be representative of functionality of control units described herein. Mobile cellular device 816 interacts with them or runs some and interacts with some. In the device 816, a communications link 813 is provided that allows the handheld device to communicate with other computing devices and under some embodiments provides a channel for receiving information automatically, such as by scanning Examples of communications link 813 include allowing communication though one or more communication protocols, such as wireless services used to provide cellular access to a network, as well as protocols that provide local wireless connections to networks.

In other examples, applications can be received on a removable Secure Digital (SD) card that is connected to an interface 815. Interface 815 and communication links 813 communicate with a processor 817 (which can also embody a processor) along a bus 819 that is also connected to memory 821 and input/output (I/O) components 823, as well as clock 825 and location system 827.

I/O components 823, in one embodiment, are provided to facilitate input and output operations and the device 816 can include input components such as buttons, touch sensors, optical sensors, microphones, touch screens, proximity sensors, accelerometers, orientation sensors and output components such as a display device, a speaker, and or a printer port. Other I/O components 823 can be used as well.

Clock 825 illustratively comprises a real time clock component that outputs a time and date. It can also provide timing functions for processor 817.

Illustratively, location system 827 includes a component that outputs a current geographical location of device 816. This can include, for instance, a global positioning system (GPS) receiver, a LORAN system, a dead reckoning system, a cellular triangulation system, or other positioning system. It can also include, for example, mapping software or navigation software that generates desired maps, navigation routes and other geographic functions.

Memory 821 stores operating system 829, network settings 831, applications 833, application configuration settings 835, data store 837, communication drivers 839, and communication configuration settings 841. Memory 821 can include all types of tangible volatile and non-volatile computer-readable memory devices. It can also include computer storage media (described below). Memory 821 stores computer readable instructions that, when executed by processor 817, cause the processor to perform computer-implemented steps or functions according to the instructions. Processor 817 can be activated by other components to facilitate their functionality as well.

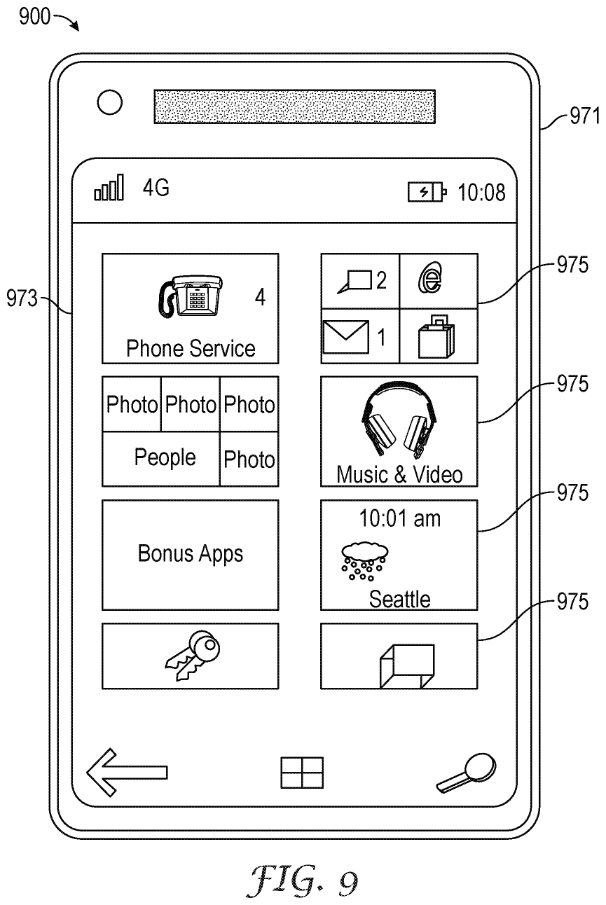

FIG. 9 shows that the device can also be a smart phone 971. Smart phone 971 has a touch sensitive display 973 that displays icons or tiles or other user input mechanisms 975. Mechanisms 975 can be used by a user to run applications, make calls, perform data transfer operations, etc. In general, smart phone 971 is built on a mobile operating system and offers more advanced computing capability and connectivity than a feature phone. Note that other forms of the devices are possible.

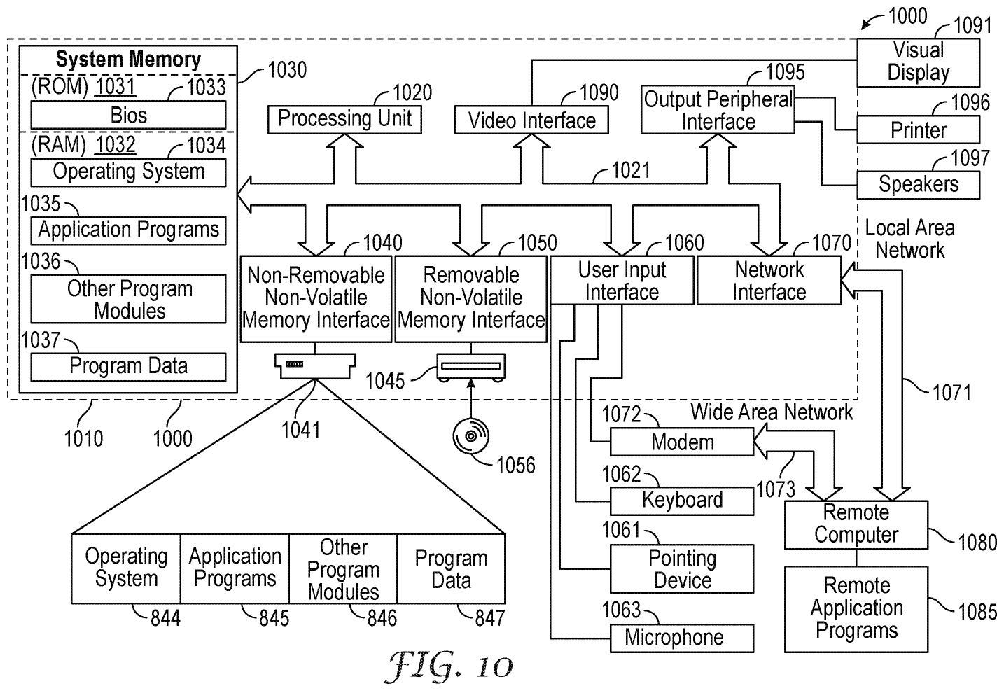

FIG. 10 is one example of a computing environment in which elements of systems and methods described herein, or parts of them (for example), can be deployed. With reference to FIG. 10, an example system for implementing some embodiments includes a general-purpose computing device in the form of a computer 1010. Components of computer 1010 may include, but are not limited to, a processing unit 1020 (which can comprise a processor), a system memory 1030, and a system bus 1021 that couples various system components including the system memory to the processing unit 1020. The system bus 1021 may be any of several types of bus structures including a memory bus or memory controller, a peripheral bus, and a local bus using any of a variety of bus architectures. Memory and programs described with respect to systems and methods described herein can be deployed in corresponding portions of FIG. 10.

Computer 1010 typically includes a variety of computer readable media. Computer readable media can be any available media that can be accessed by computer 1010 and includes both volatile/nonvolatile media and removable/non-removable media. By way of example, and not limitation, computer readable media may comprise computer storage media and communication media. Computer storage media is different from, and does not include, a modulated data signal or carrier wave. It includes hardware storage media including both volatile/nonvolatile and removable/non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 1010. Communication media may embody computer readable instructions, data structures, program modules or other data in a transport mechanism and includes any information delivery media. The term “modulated data signal” means a signal that has one or more of its characteristics set or changed in such a manner as to encode information in the signal.

The system memory 1030 includes computer storage media in the form of volatile and/or nonvolatile memory such as read only memory (ROM) 1031 and random-access memory (RAM) 1032. A basic input/output system 1033 (BIOS) containing the basic routines that help to transfer information between elements within computer 1010, such as during start-up, is typically stored in ROM 1031. RAM 1032 typically contains data and/or program modules that are immediately accessible to and/or presently being operated on by processing unit 1020. By way of example, and not limitation, FIG. 10 illustrates operating system 1034, application programs 1035, other program modules 1036, and program data 1037.

The computer 1010 may also include other removable/non-removable and volatile/nonvolatile computer storage media. By way of example only, FIG. 10 illustrates a hard disk drive 1041 that reads from or writes to non-removable, nonvolatile magnetic media, nonvolatile magnetic disk 1052, an optical disk drive 1055, and nonvolatile optical disk 1056. The hard disk drive 1041 is typically connected to the system bus 1021 through a non-removable memory interface such as interface 1040, and optical disk drive 1055 are typically connected to the system bus 1021 by a removable memory interface, such as interface 1050.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (e.g., ASICs), Application-specific Standard Products (e.g., ASSPs), System-on-a-chip systems (SOCs), Complex Programmable Logic Devices (CPLDs), etc.

The drives and their associated computer storage media discussed above and illustrated in FIG. 10, provide storage of computer readable instructions, data structures, program modules and other data for the computer 1010. In FIG. 10, for example, hard disk drive 1041 is illustrated as storing operating system 844, application programs 845, other program modules 846, and program data 847. Note that these components can either be the same as or different from operating system 1034, application programs 1035, other program modules 1036, and program data 1037.

A user may enter commands and information into the computer 1010 through input devices such as a keyboard 1062, a microphone 1063, and a pointing device 1061, such as a mouse, trackball or touch pad. Other input devices (not shown) may include a joystick, game pad, satellite receiver, scanner, or the like. Another input device may include a video camera built into a headset or other device that can capture movement of a user, such that gestures can be used as inputs. Similarly, for hearing protection devices that incorporate an in-ear sound sensor, physical movements of a user may be picked up as inputs, such as tongue clicks, teeth clicks or blinking movements. These and other input devices are often connected to the processing unit 1020 through a user input interface 1060 that is coupled to the system bus but may be connected by other interface and bus structures. A visual display 1091 or other type of display device is also connected to the system bus 1021 via an interface, such as a video interface 1090. In addition to the monitor, computers may also include other peripheral output devices such as speakers 1097 and printer 1096, which may be connected through an output peripheral interface 1095.

The computer 1010 is operated in a networked environment using logical connections, such as a Local Area Network (LAN) or Wide Area Network (WAN) to one or more remote computers, such as a remote computer 1080.

When used in a LAN networking environment, the computer 1010 is connected to the LAN 1071 through a network interface or adapter 1070. When used in a WAN networking environment, the computer 1010 typically includes a modem 1072 or other means for establishing communications over the WAN 1073, such as the Internet. In a networked environment, program modules may be stored in a remote memory storage device. FIG. 10 illustrates, for example, that remote application programs 1085 can reside on remote computer 1080.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example. The techniques of this disclosure may be implemented in a wide variety of computer devices, such as servers, laptop computers, desktop computers, notebook computers, tablet computers, hand-held computers, smart phones, and the like. Any components, modules or units have been described to emphasize functional aspects and do not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset. Additionally, although a number of distinct modules have been described throughout this description, many of which perform unique functions, all the functions of all of the modules may be combined into a single module, or even split into further additional modules. The modules described herein are only exemplary and have been described as such for better ease of understanding.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

A hearing protection assessment module for a Personal Protective Equipment (PPE) device is presented that includes an ambient sound receiver that receives an indication of an ambient sound. The module also includes an experienced sound receiver that receives an indication of an experienced sound. The experienced sound is a sound experienced by a user while wearing the PPE in the environment with the ambient sound. The module also includes a Field Attenuation Rating (FAR) calculator that, based on the ambient and attenuated sound indications, calculates a personal attenuation rating for the PPE device.

The module may also be implemented such that a controller that, when triggered, causes the ambient sound receiver to capture a current indication of ambient sound, the attenuated sound receiver to capture a current indication of experienced sound, and causes the FAR calculator to calculate a current personal attenuation rating based on the current ambient and experienced sound indications.

The module may also be implemented such that the current ambient sound indication and the current experienced sound indication are captured substantially simultaneously.

The module may also be implemented such that the controller is triggered periodically.

The module may also be implemented such that the controller is triggered based on an indication that the PPE is in use by a user.

The module may also be implemented such that the controller is triggered by an accelerometer indication of movement by the PPE.

The module may also be implemented such that the controller is triggered manually.

The module may also be implemented such that the controller is triggered by a detected change in an ambient sound level.

The module may also be implemented such that it includes electronics configured to receive an ambient sound from a microphone, process the ambient sound, and provides the sound to a speaker of the PPE. The experienced sound is a processed sound generated by a device of the PPE to a safe sound level. The experienced sound receiver receives the indication of the experienced sound from a receiver located on an interior of the PPE device.

The module may also be implemented such that processing the ambient sound includes applying a level dependent function.

The module may also be implemented such that processing the ambient sound includes applying an active noise reduction to the received sound.

The module may also be implemented such that it includes an attenuation receiver that receives an indication of an expected attenuation level. The FAR calculator provides an indication of a deviation of the current attenuated sound from the expected attenuation level.

The module may also be implemented such that the deviation indication is proved to a remote storage.

The module may also be implemented such that the deviation indication is provided to a wearer of the PPE.

The module may also be implemented such that the deviation indication is provided audibly to the user through an audio speaker within the PPE in fluid communication with the user.

The module may also be implemented such that it includes a communication component configured to provide the deviation indication to a remote device.

The module may also be implemented such that the remote device includes a memory configured to store a plurality of deviation indications received from the communication component.

The module may also be implemented such that the controller is triggered by a signal received from a remote device.

The module may also be implemented such that the FAR is converted to a PAR value using a field diffuse transformation.

A hearing protection device includes a pair of ear components. Each ear component includes a sound receiver configured to capture an ambient sound, a speaker configured to broadcast an attenuated sound, and a sealing feature configured to at least partially seal an inner portion of the ear component from an outside of the ear component. The attenuated sound is broadcast in the inner portion. The hearing protection device also includes an experienced sound receiver positioned in the inner portion, configured to capture an indication of an experienced sound, experienced by a wearer of the pair of ear components in an environment with the ambient sound. The hearing protection device also includes a field attenuation rating processor configured to, based on the indication of the ambient sound and the indication of the experienced sound, calculate a field attenuation rating (FAR) value for the hearing protection device.

The hearing protection device may also be implemented such that the indication of ambient sound is an ambient sound pressure. The indication of attenuated sound is an attenuated sound pressure.

The hearing protection device may also be implemented such that the FAR value is based on a difference between the attenuated sound level and the ambient sound level.

The hearing protection device may also be implemented such that the FAR value is a PAR value.

The hearing protection device may also be implemented such that it includes an attenuation processor configured to take the ambient sound and apply a filter to obtain the attenuated sound.

The hearing protection device may also be implemented such that the filter is a level dependent function.

The hearing protection device may also be implemented such that the filter is an active noise reduction function.

The hearing protection device may also be implemented such that a communication component is configured to communicate the FAR value to a second device.

The hearing protection device may also be implemented such that the personal attenuation rating processor provides a timestamp with the calculated FAR value.

The hearing protection device may also be implemented such that an alert is provided if the calculated FAR value exceeds a threshold.

The hearing protection device may also be implemented such that the second device stores the FAR value.

The hearing protection device may also be implemented such that based on the FAR value, an effectiveness indication of the sealing feature is provided.

The hearing protection device may also be implemented such that the effectiveness indication is an indication of proper fit.

The hearing protection device may also be implemented such that it includes a power source that, based on a command from the personal attenuation rating processor, powers the ambient and attenuated sound receivers.

The hearing protection device may also be implemented such that the personal attenuation rating processor sends the command periodically.

The hearing protection device may also be implemented such that the personal attenuation rating processor sends the command based on a manual input.

The hearing protection device may also be implemented such that the command is sent at a start-up of the hearing protection device.

The hearing protection device may also be implemented such that the ambient and experienced sound receivers operate substantially continuously.

The hearing protection device may also be implemented such that the ambient and experienced sound receivers capture indications substantially simultaneously.

The hearing protection device may also be implemented such that the ear components are earmuffs.

The hearing protection device may also be implemented such that the pair of earmuffs are coupled by a headband.

The hearing protection device may also be implemented such that the pair of earmuffs are coupled to a helmet.

The hearing protection device may also be implemented such that the pair of earmuffs are coupled by a connector configured to be worn behind a neck of a user.

The hearing protection device may also be implemented such that the ear components are earplugs.

The hearing protection device may also be implemented such that the ear components are a first set of ear components enabled for dual-hearing protection when used in combination with a second set of ear component. One of the first and second ear components is an in-ear hearing protection device. One of the first and second ear components is an over-ear hearing protection device.

The hearing protection device may also be implemented such that it includes a control unit that houses the FAR processor.

The hearing protection device may also be implemented such that the filter reduces sound above a threshold sound level.

A method of evaluating hearing protection of a personal protective equipment device that includes activating a personal attenuation rating monitoring module of a hearing protection system. The hearing protection system includes a passive sound attenuation feature and a seal that separates an ear canal of a user from an ambient environment. The method also includes calculating a FAR value, using a FAR calculator, for the active hearing protection device in the hearing protection system. Calculating includes retrieving an ambient sound signal from an ambient sound receiver and an experienced sound signal, from an experienced sound receiver and comparing the experienced sound to the ambient sound. The FAR value is an indication of the comparison. The method also includes providing an indication of the FAR value.

The method may also be implemented such that the comparison includes an indication of a difference between the ambient sound signal and the experienced sound signal.

The method may also be implemented such that the comparison further includes a diffuse field correction.

The method may also be implemented such that it includes calculating an attenuation deviation. The attenuation indication includes a comparison of the calculated FAR to an expected attenuation.

The method may also be implemented such that it includes providing an alert if the attenuation deviation is greater than a threshold.

The method may also be implemented such that the hearing protection system includes a microphone configured to capture an ambient sound, and a speaker configured to broadcast the experienced sound.

The method may also be implemented such that the microphone is on an outer portion of the hearing protection device. The speaker is on an inner portion of the hearing protection device.

The method may also be implemented such that the experienced sound is a processed sound. The processing includes applying a level dependent function to the captured ambient sound.

The method may also be implemented such that the experienced sound is a processed sound. The processing includes applying an active noise reduction function to the captured ambient sound.

The method may also be implemented such that the PPE is an earmuff. The speaker is on an interior of the earmuff.

The method may also be implemented such that the PPE is an earplug device or in-ear headset. The speaker is positioned to broadcast into an ear canal.

The method may also be implemented such that the hearing protection system includes a control unit communicably coupled to the hearing protection device.

The method may also be implemented such that the FAR calculator is housed within the control unit. The indication is provided from the control unit to the hearing protection device.

The method may also be implemented such that the control unit provides the FAR value to a storage.

The method may also be implemented such that the steps of calculating and providing are repeated periodically.

A user safety monitoring system includes a field attenuation rating (FAR) value request generator that generates a FAR value request. The system also includes a communications component that transmits the FAR value request to a PPE device worn by a user in an environment. The system also includes a FAR value receiver that receives, from the PPE device, a FAR notification. The FAR notifications include a FAR value, a PPE device identification, a timestamp, and a memory component that stores the received FAR notification.

The system may also be implemented such that the FAR request generator generates the FAR value request periodically.

The system may also be implemented such that the FAR request generator generates the FAR value at start-up.

The system may also be implemented such that the FAR request generator generates the FAR value in response to a manual input from the user.

The system may also be implemented such that a FAR evaluator that compares the FAR value to a threshold and, if the FAR value exceeds or does not meet a threshold, an alert generator generates an alert.

The system may also be implemented such that the FAR evaluator provides an indication of PPE fit based on the comparison.

The system may also be implemented such that the PPE device is an active hearing protection device.

The system may also be implemented such that the active hearing protection device includes a headset.

The system may also be implemented such that the active hearing protection device includes a pair of earbuds.

The system may also be implemented such that the FAR notification includes sensor signals from an inner sensor, on an interior of the active hearing protection device, and an outer sensor, on a housing of the active hearing protection device.

The system may also be implemented such that the communication device transmits the FAR value request to a control unit associated with the PPE device. The control unit transmits the FAR notification to the system.

The system may also be implemented such that the FAR value is a PAR value.

The present invention has now been described with reference to several embodiments thereof. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the exact details and structures described herein, but rather by the structures described by the language of the claims, and the equivalents of those structures. Any patent literature cited herein is hereby incorporated herein by reference in its entirety to the extent that it does not conflict with the description presented herein.

Any feature or characteristic described with respect to any of the above embodiments can be incorporated individually or in combination with any other feature or characteristic, and are presented in the above order and combinations for clarity only. That is, the present disclosure contemplates all possible combinations and arrangements of various features of each of the exemplary embodiments and components described herein, and each component may be combined or used in conjunction with any other component as may be desired for a particular application.

1. A hearing protection assessment module for a Personal Protective Equipment (PPE) device, comprising:
an ambient sound receiver that receives an indication of an ambient sound;
an experienced sound receiver that receives an indication of an experienced sound, wherein the experienced sound is a sound experienced by a user while wearing the PPE in the environment with the ambient sound;
a Field Attenuation Rating (FAR) calculator that, based on the ambient and attenuated sound indications, calculates a personal attenuation rating for the PPE device.

2. The module of claim 1, and further comprising a controller that, when triggered, causes the ambient sound receiver to capture a current indication of ambient sound, the attenuated sound receiver to capture a current indication of experienced sound, and causes the FAR calculator to calculate a current personal attenuation rating based on the current ambient and experienced sound indications.

3. The module of claim 2, wherein the current ambient sound indication and the current experienced sound indication are captured substantially simultaneously.

4. The module of claim 1, and further comprising:
electronics configured to receive an ambient sound from a microphone, process the ambient sound, and provides the sound to a speaker of the PPE;
wherein the experienced sound is a processed sound generated by a device of the PPE to a safe sound level; and
wherein the experienced sound receiver receives the indication of the experienced sound from a receiver located on an interior of the PPE device.

5. The module of claim 1, and further comprising:
an attenuation receiver that receives an indication of an expected attenuation level; and
wherein the FAR calculator provides an indication of a deviation of the current attenuated sound from the expected attenuation level.

6-9. (canceled)

10. The module of claim 1, wherein the FAR is converted to a PAR value using a field diffuse transformation.

11. A hearing protection device comprising:
a pair of ear components, each ear component comprising:
a sound receiver configured to capture an ambient sound;
a speaker configured to broadcast an attenuated sound; and
a sealing feature configured to at least partially seal an inner portion of the ear component from an outside of the ear component, wherein the attenuated sound is broadcast in the inner portion;
an experienced sound receiver positioned in the inner portion, configured to capture an indication of an experienced sound, experienced by a wearer of the pair of ear components in an environment with the ambient sound;
a field attenuation rating processor configured to, based on the indication of the ambient sound and the indication of the experienced sound, calculate a field attenuation rating (FAR) value for the hearing protection device.

12. The hearing protection device of claim 11, wherein the indication of ambient sound is an ambient sound pressure, and wherein the indication of attenuated sound is an attenuated sound pressure.

13. (canceled)

14. (canceled)

15. (canceled)

16. The hearing protection device of claim 11, wherein, based on the FAR value, an effectiveness indication of the sealing feature is provided.

17. The hearing protection device of claim 11, and further comprising a power source that, based on a command from the personal attenuation rating processor, powers the ambient and attenuated sound receivers.

18-21. (canceled)

22. The hearing protection device of claim 11, wherein the filter reduces sound above a threshold sound level.

23. A method of evaluating hearing protection of a personal protective equipment device, the method comprising:
activating a personal attenuation rating monitoring module of a hearing protection system, wherein the hearing protection system comprises a passive sound attenuation feature and a seal that separates an ear canal of a user from an ambient environment;
calculating a FAR value, using a FAR calculator, for the active hearing protection device in the hearing protection system, wherein calculating comprises retrieving an ambient sound signal from an ambient sound receiver and an experienced sound signal, from an experienced sound receiver and comparing the experienced sound to the ambient sound, wherein the FAR value is an indication of the comparison; and
providing an indication of the FAR value.

24. The method of claim 23, wherein the comparison comprises an indication of a difference between the ambient sound signal and the experienced sound signal.

25. The method of claim 24, wherein the comparison further comprises a diffuse field correction.

26. (canceled)

27. The method of claim 23, wherein the hearing protection system comprises a microphone configured to capture an ambient sound, and a speaker configured to broadcast the experienced sound.

28. The method of claim 27, wherein the microphone is on an outer portion of the hearing protection device, and wherein the speaker is on an inner portion of the hearing protection device.

29. The method of claim 28, wherein the experienced sound is a processed sound, and wherein the processing comprises applying a level dependent function to the captured ambient sound.

30. The method of claim 28, wherein the experienced sound is a processed sound, and wherein the processing comprises applying an active noise reduction function to the captured ambient sound.

31. The method of claim 23, wherein the hearing protection system comprises a control unit communicably coupled to the hearing protection device.

32. (canceled)

33. The method of claim 23, wherein the steps of calculating and providing are repeated periodically.

34-43. (canceled)
